(19) United States
(12) Patent Application Publication
(10) Pub. No.: US 2013/0149275 A1
(43) Pub. Date: Jun. 13, 2013

(54) AGENTS FOR TEMPORARY SHAPING OF KERATINOUS FIBERS

(71) Applicant: Henkel AG & Co., KGaA, Duesseldorf (DE)

(72) Inventors: Burkhard Muller, Hamburg (DE); Pamela Kaftan, Hamburg (DE)

(73) Assignee: HENKEL AG & CO. KGAA, Duesseldorf (DE)

(21) Appl. No.: 13/760,102

(22) Filed: Feb. 6, 2013

Related U.S. Application Data
(63) Continuation of application No. PCT/EP2011/064963, filed on Aug. 31, 2011.

(30) Foreign Application Priority Data
Sep. 23, 2010 (DE) 102010041267.8

(51) Int. Cl. A61K 8/81; A61Q 5/02

(52) U.S. Cl. 424/70.15; 424/70.16

(57) ABSTRACT

An agent for treating keratin-containing fibers includes in a cosmetic carrier: (a) a cationic cellulose compound; (b) a copolymer containing a structural unit of Formula (I), a structural unit of Formula (II), and a structural unit of Formula (III) in which R¹ is a hydrogen atom or a methyl group, X¹ is an oxygen atom or an NH group, R² and R³ independently of one another are each a (C₁ to C₄) alkyl group; and (c) a copolymer containing a structural unit of Formula (I) and a structural unit of Formula (IV) in which R⁴ is a hydrogen atom or a methyl group, X² is an oxygen atom or an NH group, A is a (C₂ to C₃) alkylene group, and R⁵ and R⁶ independently of one another are each a (C₁ to C₄) alkyl group. Methods for treating keratin-containing fibers using the agents are also provided.

### CROSS-REFERENCE TO RELATED APPLICATION

This is a continuation of International Application No. PCT/EP2011/064963 filed Aug. 31, 2011, which was published under PCT Article 21(2) and which claims priority to German Application No. 102010041267.8, filed Sep. 23, 2010, which are all hereby incorporated in their entirety by reference.

### TECHNICAL FIELD

The various embodiments relate to agents for the temporary shaping of keratin-containing fibers, comprising in a cosmetic carrier a combination of a cationic cellulose compound, a copolymer that contains pyrrolidone, caprolactam and dialkylaminoethyl groups and a copolymer that differs from that copolymer and which contains pyrrolidone and dialkylaminoalkyl groups, the use of these agents for the temporary shaping and/or for the care of keratin-containing fibers, and aerosol hair sprays/foams based on these agents.

### BACKGROUND

Today, a suitably looking hairstyle is generally regarded as an essential part of a well groomed appearance. Based on current fashion trends, time and again hairstyles are considered chic, which, for many types of hair, can only be formed or sustained over a longer period of up to several days by the use of certain setting materials. Thus, hair treatment agents, which provide a permanent or temporary hairstyling, play an important role. Temporary styling intended to provide a good hold, without compromising the healthy appearance of the hair, such as, for example the gloss, can be obtained for example by the use of hairsprays, hair waxes, hair gels, hair foams, setting lotions etc.

Suitable compositions for temporary hairstyling usually comprise synthetic polymers as the styling component. Preparations comprising a dissolved or dispersed polymer can be applied on the hair by means of propellants or by a pumping mechanism. Hair gels and hair waxes in particular are however not generally applied directly on the hair, but rather dispersed with a comb or by hand.

The most important property of an agent for the temporary styling of keratin fibers, in the following also called styling agents, consists in giving the treated fibers the strongest possible hold in the created shape. If the keratinic fibers concern human hair, then one also speaks of a strong hairstyle hold or a high degree of hold of the styling agent. The styling hold is determined essentially by the type and quantity of the synthetic polymer used, but there may also be an influence from the other components of the styling agent.

In addition to a high degree of hold, styling agents typically fulfill a whole series of additional requirements. These requirements can be broadly subdivided into properties on the hair, properties of the formulation in question, e.g. properties of the foam, the gel or the sprayed aerosol, and properties that concern the handling of the styling agent, wherein particular importance is attached to the properties on the hair. In particular, moisture resistance, low stickiness and a balanced conditioning effect should be mentioned. Furthermore, a styling agent should be universally applicable for as many types of hair as possible and be mild to hair and skin.

To do justice to the various requirements, various synthetic polymers have already been developed and are being used in styling agents. The polymers can be subdivided into cationic, anionic, non-ionic and amphoteric setting polymers. Ideally these polymers form a polymer film when applied to hair, imparting on the one hand a strong hold to the hairstyle but on the other hand also being sufficiently flexible not to break under stress. If the polymer film is too brittle, so-called film plaques develop, i.e. residues that are shed with movement of the hair and give the impression that the user of the respective styling agent has dandruff.

To develop styling agents that in combination have all the desired properties still presents problems. This is particularly true for the combination of a strong hold on the one hand and on the other hand a simple, uniform application onto the keratin-containing fibers.

Accordingly, it is desirable to provide an agent for the temporary styling of keratinic fibers, which is characterized by a very high degree of hold. Furthermore, it is desirable to provide an agent that shows a good effect on volume as well as being highly conditioning. The formation of film plaques—in particular the formation of corresponding residues during the application, on brushing and when drying the keratin-containing fibers—should be avoided. In addition, other objects, desirable features and characteristics will become apparent from the subsequent summary and detailed description, and the appended claims, taken in conjunction with the accompanying drawings and this background.

### SUMMARY

It has now been surprisingly found that an agent with the above properties can be achieved by a combination of a cationic cellulose compound with a copolymer containing pyrrolidon-N-yl, caprolactam-N-yl and dialkylaminoethyl groups and a copolymer that differs from that copolymer and which contains pyrrolidone-N-yl and dialkylaminoalkyl groups. The compositions obtained with this combination even exhibit—when they are formulated as a hair foam—particularly good foam parameters.

### DETAILED DESCRIPTION

Accordingly, an exemplary embodiment includes an agent for treating keratin-containing fibers, especially human hair, comprising in a cosmetic carrier

(a) a cationic cellulose compound
and
(b) a copolymer containing a structural unit of the Formula (I) and a structural unit of the Formula (II) and a structural unit of the Formula (III)

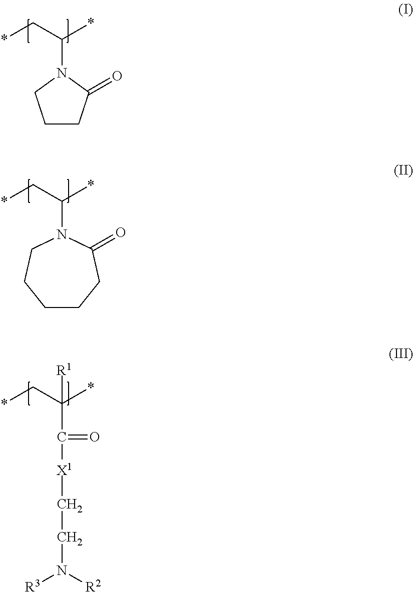

in which
R¹ is a hydrogen atom or a methyl group,
X¹ is an oxygen atom or an NH group,
R² and R³ independently of one another are each a (C₁ to C₄) alkyl group
and
(c) a copolymer containing a structural unit of Formula (I) and a structural unit of Formula (IV)

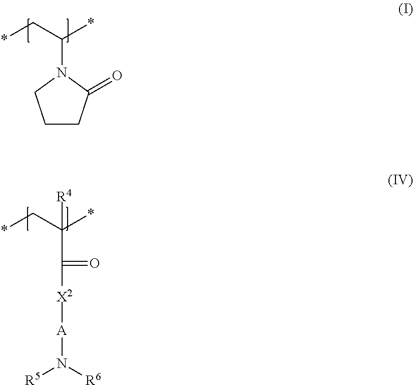

in which
R⁴ is a hydrogen atom or a methyl group,
X² is an oxygen atom or an NH group,
A is a (C₂ to C₃) alkylene group (in particular ethane-1,2-diyl or propane-1,3-diyl),
R⁵ and R⁶ independently of one another are each a (C₁ to C₄) alkyl group.

Keratin-containing fibers are in principle understood to include all animal hair, e.g. wool, horsehair, angora hair, furs, feathers and products or fabrics produced from them. However, the keratinic fibers preferably concern human hair.

The compounds of the components (a), (b) and (c) comprised in the inventive agent differ from one another.

In the above Formulas and all Formulas below, the symbol * signifies a chemical bond that stands for a free valence of the corresponding structural fragment.

Those cationic cellulose compounds that carry more than one permanent cationic charge in a side chain have proven to be preferred and particularly advantageous in the context herein. Cellulose is based on β-1,4-glycosidically linked D-glucopyranose units and forms un-branched, water-insoluble chains. The side chain of a cellulose is defined as chemical substituents that bond to the cellulose backbone and which are not found in native cellulose, because they have been subsequently introduced e.g. by chemical synthesis.

Among the cationic cellulose derivatives that are to be highlighted are those produced from the reaction of hydroxy(C₂ to C₄) alkyl cellulose with a dimethyldiallylammonium reactant (especially dimethyldiallylammonium chloride) optionally in the presence of further reactants. Among these cationic celluloses, once again those cationic celluloses with the INCI name Polyquaternium-4 are particularly suitable, which, for example, are marketed by the National Starch Company under the trade names Celquat® H 100 and Celquat® L 200.

The agents as contemplated herein preferably comprise the cationic cellulose compounds (a) in an amount of about 0.01 wt % to about 3.0 wt %, for example about 0.4 wt % to about 1.8 wt %, for example about 0.8 wt % to about 1.2 wt %, each based on the weight of the agent.

In an exemplary embodiment, the agents include a copolymer (b) that according to Formula (III) fulfills at least one of the following parameters (particularly preferably all three together):
R¹ is a methyl group
X¹ is an oxygen atom,
R² and R³ are each a methyl group.

In another embodiment, the inventive agent comprises as the copolymer (b) a terpolymer obtained from N-vinyl pyrrolidone, N-vinyl caprolactam and N,N-dimethylaminoethyl methacrylate. Such copolymers can be ordered for example from the ISP Company under the trade name Advantage S (INCI name: Vinylcaprolactam/VP/Dimethyl-aminoethylmethacrylate Copolymer, in powder form), Advantage LC-E (INCI name: Vinylcaprolactam/VP/Dimethylaminoethylmethacrylate Copolymer, Laurylpyrrolidone; 37 wt % active substance in ethanol with added N-lauryl pyrrolidone) or Advantage LC-A (INCI name: Vinylcaprolactam/VP/Dimethylaminoethylmethacrylate Copolymer; 37 wt % active substance in ethanol).

The agents in an embodiment include the copolymers (b) in an amount of about 0.01 wt % to about 10.0 wt %, for example about 0.5 wt % to about 8.0 wt %, such as about 0.5 wt % to about 5.0 wt %, each based on the weight of the agent.

In another embodiment, the agent contemplated herein comprises such a copolymer (c) that according to Formula (IV) fulfills at least one of the following parameters (particularly preferably all four together):
R⁴ is a methyl group,
X² is an oxygen atom,
A is an ethane-1,2-yl group,
R⁵ and R⁶ are each a methyl group.

If the X² group according to Formula (IV) is an oxygen atom then, in an embodiment, the copolymer additionally contains a structural unit of the Formula (V)

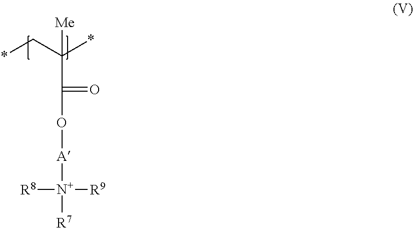

in which
R⁸ and R⁹ independently of one another are a (C₁ to C₆) alkyl group (in particular for methyl), A′ is ethane-1,2-diyl or propane-1,3-diyl (in particular ethane-1,2-diyl) and R⁷ is a (C₁ to C₄) alkyl group (in particular for ethyl).

At least one physiologically acceptable anion, such as for example chloride, bromide, hydrogen sulfate, methyl sulfate, ethyl sulfate, tetrafluoroborate, phosphate, hydrogen phosphate, dihydrogen phosphate or p-toluene sulfonate, triflate serves to compensate for the positive polymer charge of the structural unit of the Formula (V) in the agent (and when not otherwise stated, also for all further cationic compounds of the agent).

The inventive agent of this embodiment comprises as the copolymer (c) a copolymer that is obtained from N-vinyl pyrrolidone and N,N-dimethylaminoethyl methacrylate that was cationized with diethyl sulfate. Such copolymers fall under the INCI name Polyquaternium-11. They can be ordered for example from ISP under the trade name Gafquat 755 or Gafquat 734 or from BASF SE under the trade name Luviquat PQ 11 PN.

In the context of another embodiment, the agent as contemplated herein comprises such a copolymer (c) that according to Formula (IV) fulfils at least one of the following parameters (particularly preferably all four together):
R⁴ is a methyl group,
X² is an NH group,
A is a propane-1,3-diyl group,
R⁵ and R⁶ are a methyl group.

The inventive agent of this embodiment comprises as the copolymer (c) a copolymer that is obtained from N-vinyl pyrrolidone and N,N-dimethylaminopropyl methacrylamide. Such copolymers fall under the INCI name VP/DMAPA Acrylates copolymer. They can be ordered for example from ISP under the trade name Styleze CC (ca. 10 wt % polymer solution in water).

Consequently, in an embodiment, if in Formula (IV)

R⁴ is a methyl group,
X² is an oxygen atom or an NH group,
A is ethane-1,2-diyl or propane-1,3-diyl and
R⁵ and R⁶ independently of one another are each a (C₁ to C₄) alkyl group (in particular methyl), with the proviso that
when X² is an oxygen atom, then A is ethane-1,2-diyl, and
when X² is an NH group, then A is propane-1,3-diyl.

The agents contemplated herein comprise the copolymers (c) in an amount of about 0.01 wt % to about 5.0 wt %, for example about 0.1 wt % to about 2.0 wt %, such as about 0.15 wt % to about 1.5 wt %, each based on the weight of the agent.

In a further embodiment, an agent comprises in a cosmetic carrier

(a) a cationic cellulose compound produced from the reaction of hydroxy(C₂ to C₄) alkyl cellulose with a dimethyldiallylammonium reactant (especially dimethyldiallylammonium chloride) optionally in the presence of further reactants
and
(b) a copolymer containing a structural unit of the Formula (I) and a structural unit of the Formula (II) and a structural unit of the Formula (III)

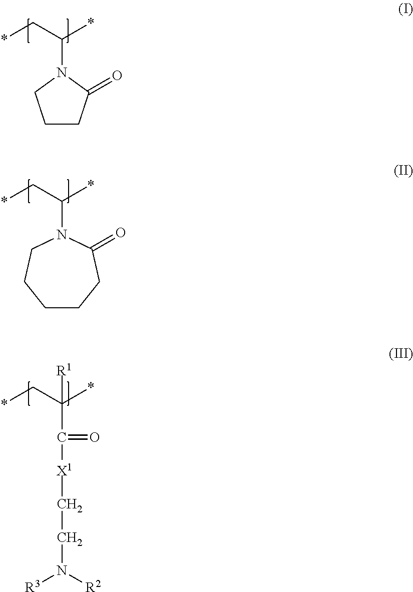

where R¹ is a methyl group, X¹ is an oxygen atom and R² and R³ are methyl groups
and
(c) a copolymer containing a structural unit of Formula (I) and a structural unit of Formula (IV)

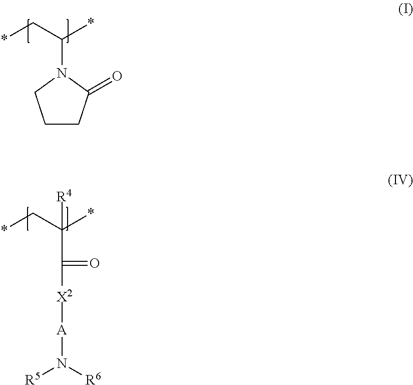

in which
R⁴ is a hydrogen atom or a methyl group,
X² is an oxygen atom or an NH group,
A is a (C₂ to C₃) alkylene group (in particular ethane-1,2-diyl or propane-1,3-diyl),
R⁵ and R⁶ independently of one another are each a (C₁ to C₄) alkyl group.

In yet another embodiment, an agent comprises in a cosmetic carrier

(a) a cationic cellulose compound produced from the reaction of hydroxy(C₂ to C₄) alkyl cellulose with a dimethyldiallylammonium reactant (especially dimethyldiallylammonium chloride) optionally in the presence of further reactants.
and
(b) a copolymer containing a structural unit of the Formula (I) and a structural unit of the Formula (II) and a structural unit of the Formula (III)

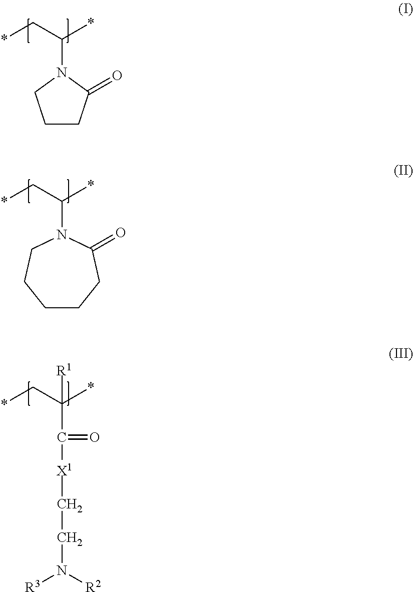

where
R¹ is a methyl group, X¹ is an oxygen atom and R² and R³ are methyl groups
and
(c) a copolymer containing a structural unit of Formula (I) and a structural unit selected from Formula (IV)

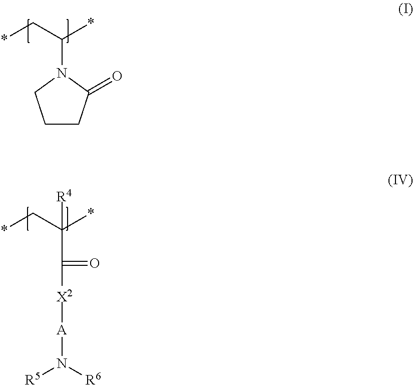

in which
R⁴ is a methyl group,
X² is an oxygen atom or an NH group,
A is ethane-1,2-diyl or propane-1,3-diyl and
R⁵ and R⁶ independently of one another are each a (C₁ to C₄) alkyl group, with the proviso that when X² is an oxygen atom, then A is ethane-1,2-diyl, and
when X² is an NH group, then A is propane-1,3-diyl.

In yet a further embodiment, an agent comprises in a cosmetic carrier

(a) a cationic cellulose compound of the INCI name Polyquaternium-4 and
(b) a copolymer of the INCI name Vinylcaprolactam/VP/Dimethylaminoethyl-methacrylate copolymer
(c) a copolymer selected from a copolymer of the group that is formed of copolymers of the INCI name Polyquaternium-11 and copolymers of the INCI name VP/DMAPA Acrylates Copolymer.

INCI (INCI=International Nomenclature of Cosmetic Ingredients) refers to an international guideline for the correct and uniform description of cosmetic ingredients. A name of a cosmetic ingredient or of a group of cosmetic ingredients retrieved from INCI is inventively defined as the INCI name.

The following are embodiments (A) to (D) of agents contemplated herein:

An agent for treating keratin-containing fibers, especially human hair, comprising in a cosmetic carrier
(a) about 0.1 to about 3.0 wt % of one or more cationic cellulose compounds
and
(b) about 0.1 to 10 wt % of a copolymer containing a structural unit of the Formula (I) and a structural unit of the Formula (II) and a structural unit of the Formula (III)

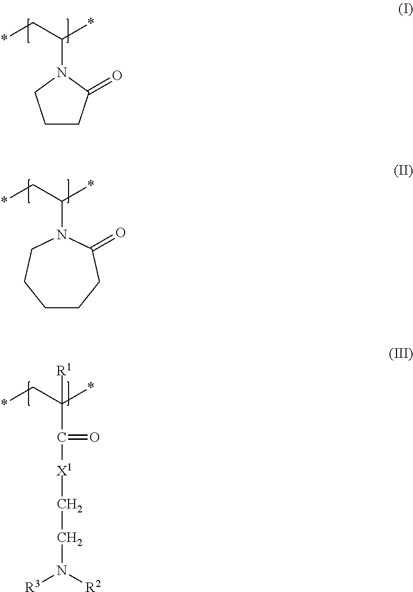

where
R¹ is a hydrogen atom or a methyl group,
X¹ is an oxygen atom or an NH group,
R² and R³ independently of one another are each a (C₁ to C₄) alkyl group
and
(c) about 0.01 to about 5.0 wt % of a copolymer containing a structural unit of the Formula (I) and a structural unit of the Formula (IV)

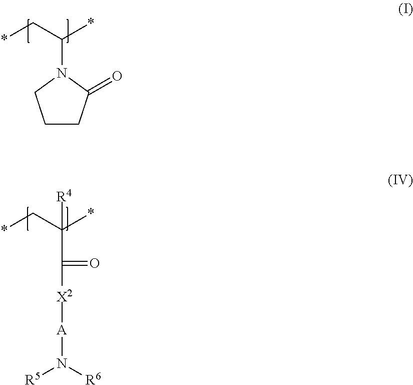

in which
R⁴ is a hydrogen atom or a methyl group,
X² is an oxygen atom or an NH group,
A is a (C₂ to C₃) alkylene group (in particular ethane-1,2-diyl or propane-1,3-diyl),
R⁵ and R⁶ independently of one another are each a (C₁ to C₄) alkyl group.

(B):

An agent for treating keratin-containing fibers, especially human hair, comprising in a cosmetic carrier

(a) about 0.8 to about 1.2 wt % of a cationic cellulose compound
and
(b) about 0.5 to about 5.0 wt % of a copolymer containing a structural unit of the Formula (I) and a structural unit of the Formula (II) and a structural unit of the Formula (III)

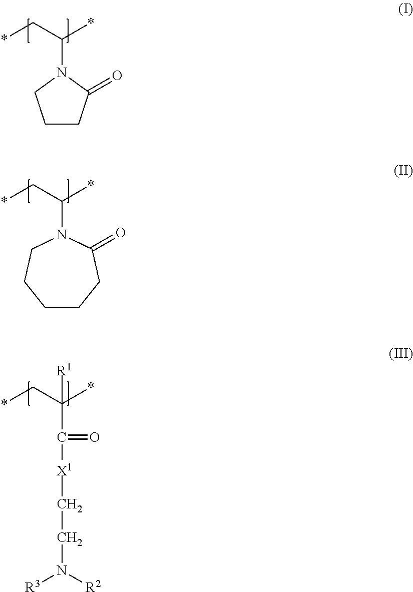

where
R¹ is a hydrogen atom or a methyl group,
X¹ is an oxygen atom or an NH group,
R² and R³ independently of one another are each a (C₁ to C₄) alkyl group,
and
(c) about 0.15 to about 1.5 wt % of a copolymer containing a structural unit of the Formula (I) and a structural unit of the Formula (IV)

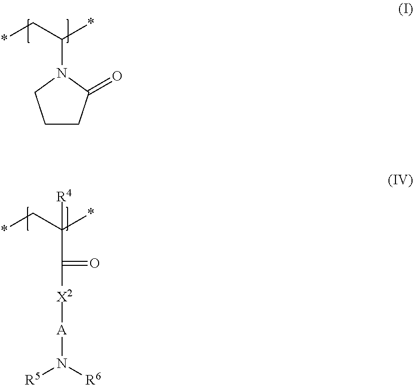

in which
R⁴ is a hydrogen atom or a methyl group,
X² is an oxygen atom or an NH group,
A is a (C₂ to C₃) alkylene group (in particular ethane-1,2-diyl or propane-1,3-diyl),
R⁵ and R⁶ independently of one another are each a (C₁ to C₄) alkyl group.

(C):

An agent for treating keratin-containing fibers, especially human hair, comprising in a cosmetic carrier

(a) about 0.1 to about 3.0 wt % of a cationic cellulose compounds of the INCI name Polyquaternium-4
and
(b) about 0.1 to about 10.0 wt % of a copolymer of the INCI name Vinylcaprolactam/VP/Dimethylaminoethyl-methacrylate Copolymer
and
(c) about 0.01 to about 5.0 wt % of a copolymer selected from a copolymer of the group that is formed of copolymers of the INCI name Polyquaternium-11 and copolymers of the INCI name VP/DMAPA Acrylates Copolymer.

(D):

An agent for treating keratin-containing fibers, especially human hair, comprising in a cosmetic carrier

(a) about 0.8 to about 1.2 wt % of a cationic cellulose compound of the INCI name Polyquaternium-4
and
(b) about 0.5 to about 5.0 wt % of a copolymer of the INCI name Vinylcaprolactam/VP/Dimethylaminoethylmethacrylate copolymer
and
(c) about 0.15 to about 1.5 wt % of a copolymer selected from a copolymer of the group that is formed of copolymers of the INCI name Polyquaternium-11 and copolymers of the INCI name VP/DMAPA Acrylates Copolymer.

The agents contemplated herein comprise the ingredients or active substances in a cosmetic carrier. Exemplary cosmetic carriers are aqueous, alcoholic or aqueous alcoholic media. In an embodiment, the inventive agent comprises at least 10 wt % water, for example at least 40 wt % water, such as at least 60 wt % water, each relative to the total agent.

In an embodiment, lower alcohols containing 1 to 4 carbon atoms, such as for example ethanol and isopropanol, which are usually used for cosmetic purposes, can be comprised as alcohols.

In the context of another embodiment, it is possible to incorporate a (C₁ to C₄) monoalkyl alcohol in the agents, for example in a quantity of about 1 to about 50 wt %, such as about 5 to about 20 wt %. Again, this is particularly preferred when manufacturing pump foams, pump sprays or aerosol foams.

Organic solvents or a mixture of solvents with a boiling point of less than 400° C. can be used as additional co-solvents in a quantity of about 0.1 to about 15 weight %, for example about 1 to 10 weight %, based on the total agent. Particularly suitable additional co-solvents are unbranched or branched hydrocarbons with more than 5 carbon atoms, such as hexane, isopentane and cyclic hydrocarbons such as cyclohexane. Further particularly suitable water-soluble solvents are glycerin, ethylene glycol and propylene glycol preferably in an amount of up to about 30 wt %, based on the total agent.

The addition of glycerin and/or propylene glycol and/or polyethylene glycol and/or polypropylene glycol increases the flexibility of the polymer film that is formed when the agent contemplated herein is used. Consequently, if a more flexible hold is desired, then the agents according to an embodiment comprise about 0.01 to about 30 wt % glycerin and/or propylene glycol and/or polyethylene glycol and/or polypropylene glycol, based on the total agent.

In addition to the cited polymers (a), (b) and (c), the agent according to an embodiment additionally comprises a cationic surfactant.

In an embodiment, the cationic surfactants are comprised in the agents in amounts of about 0.01 to about 2.0 wt %, for example about 0.1 to about 0.5 wt %.

Suitable cationic surfactants that can be inventively incorporated are of the type quaternary ammonium compounds (such as (C₈ to C₂₂) alkyltrimethylammonium compounds, di(C₈ to C₂₂) alkyldimethylammonium compounds, tri(C₈ to C₂₂) alkylmethylammonium compounds), (C₈ to C₂₂) alkyltri(oligoalkoxy)ammonium compounds, di(C₈ to C₂₂) alkyldi(oligoalkoxy)ammonium compounds of the esterquats and the amidoamines.

Particularly suitable cationic surfactants are selected from among the N—(C₁₂ to C₂₀) alkyl-N,N,N-trimethylammonium compounds, in particular their chlorides or bromides, such as in particular among cetyltrimethylammonium chloride, cetyltrimethylammonium bromide, stearyltrimethylammonium chloride, stearyltrimethylammonium bromide.

Another suitable cationic surfactant that can be incorporated possesses the general Formula (VI),

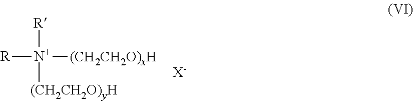

in which
x and y independently of one another are a whole number greater than 0,
R is a (C₈ to C₂₀) alkyl group or a (C₈ to C₂₀) alkenyl group,
R′ is a *—(CH₂CH₂O)_{z}H group in which z is a whole number greater than 0, a (C₈ to C₂₀) alkyl group or a (C₈ to C₂₀) alkenyl group, and
X⁻ is a physiologically compatible anion.

The agents in another embodiment comprise the cited cationic surfactant having the Formula (VI) in an amount of about 0.1 wt % to about 2 wt %, based on the weight of the agent.

In this regard, in another embodiment, the molecular weight of the cationic surfactant of Formula (VI) is from about 550 to about 2500 g/mol, for example 600 to 1000 g/mol.

In another embodiment, the R group in Formula (VI) is dodecyl, tetradecyl, hexadecyl or octadecyl.

In a further embodiment, R′ in Formula (VI) is a *—(CH₂CH₂O)_{z}H group, in which z is a whole number greater than 0.

In another embodiment, X⁻ in Formula (VI) is chloride, phosphate or dihydrogen phosphate.

Moreover, in an embodiment, in Formula (IV) x and y (and also z when R′ is a *—(CH₂CH₂O)_{z}H group) are selected, independently of one another, from a whole number selected from 1, 2, 3, 4, 5, 6, 7, 8, 9, 10.

Further, in another embodiment, when the sum of x and y in Formula (VI) (or when R′ is a *—(CH₂CH₂O)_{z}H group, the sum of x and y and z) is a number between 3 and 20, in particular a number between 5 and 15.

In one embodiment, a cationic surfactant has the Formula (Via)

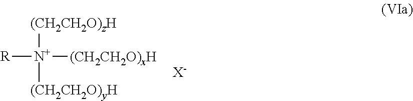

in which
x and y and z independently of one another are each a whole number greater than 0 and the sum of (x+y+z) is a number between 3 and 20, in particular between 5 and 15,
R is a (C₈ to C₂₀) alkyl group or a (C₈ to C₂₀) alkenyl group, and
X⁻ is a physiologically compatible anion.

A quite suitable cationic surfactant is the tris(oligooxyethyl)alkylammonium dihydrogen phosphate salt with a molecular weight of 780 g/mol, which is marketed with the INCI name Quaternium-52 for example under the trade name Dehyquart® SP from Cognis.

Moreover, in an exemplary embodiment, the agent contemplated herein additionally comprises, in addition to a N—(C₁₂ to C₂₂) alkyl-N,N,N-trimethylammonium compound, a compound of the above Formula (VI) (in particular of the above Formula (VIa)) as the cationic surfactant.

The following illustrated exemplary embodiments (E) to (L) of inventive agents contemplated herein:

(E):

An agent for treating keratin-containing fibers, especially human hair, comprising in a cosmetic carrier

(a) about 0.1 to about 3.0 wt % of a cationic cellulose compound
and
(b) about 0.1 to about 10 wt % of a copolymer containing a structural unit of the Formula (I) and a structural unit of the Formula (II) and a structural unit of the Formula (III)

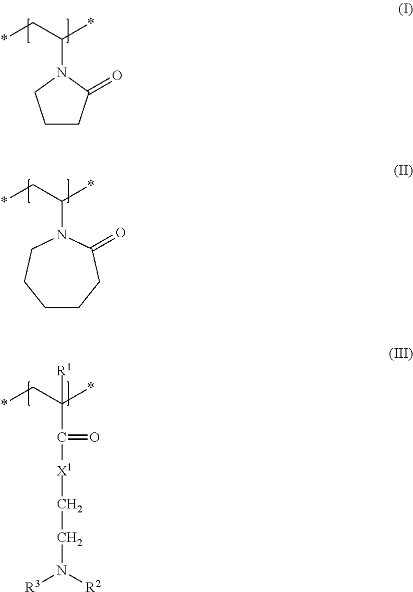

in which
R¹ is a hydrogen atom or a methyl group,
X¹ is an oxygen atom or an NH group,
R² and R³ independently of one another are each a (C₁ to C₄) alkyl group
and
(c) about 0.01 to about 5.0 wt % of a copolymer containing a structural unit of the Formula (I) and a structural unit of the Formula (IV)

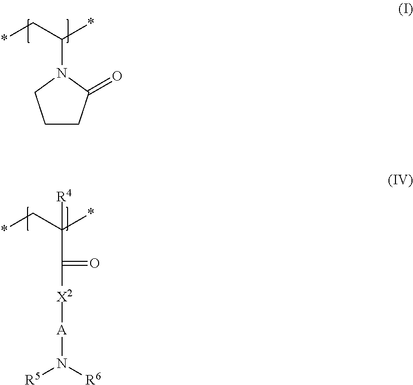

in which
R⁴ is a hydrogen atom or a methyl group,
X² is an oxygen atom or an NH group,
A is a (C₂ to C₃) alkylene group (in particular ethane-1,2-diyl or propane-1,3-diyl),
R⁵ and R⁶ independently of one another are each a (C₁ to C₄) alkyl group, and
(d) a cationic surfactant selected from N—(C₁₂ to C₂₂) alkyl-N,N,N-trimethylammonium compounds.

(F):

An agent for treating keratin-containing fibers, especially human hair, comprising in a cosmetic carrier

(a) about 0.8 to about 1.2 wt % of a cationic cellulose compound
and
(b) about 0.5 to about 5.0 wt % of a copolymer containing a structural unit of the Formula (I) and a structural unit of the Formula (II) and a structural unit of the Formula (III)

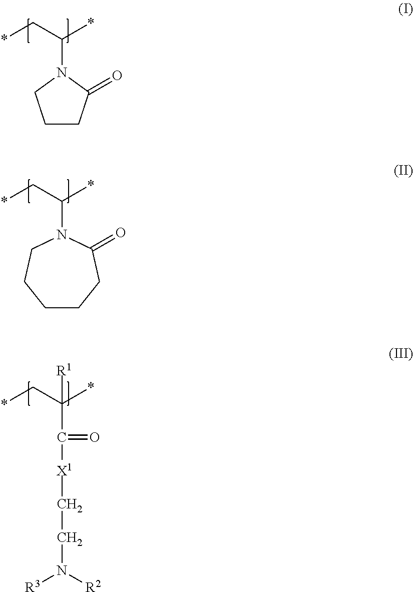

in which

R¹ is a hydrogen atom or a methyl group,
X¹ is an oxygen atom or an NH group,
R² and R³ independently of one another are each a (C₁ to C₄) alkyl group and
(c) about 0.15 to about 1.5 wt % of a copolymer containing a structural unit of the Formula (I) and a structural unit of the Formula (IV)

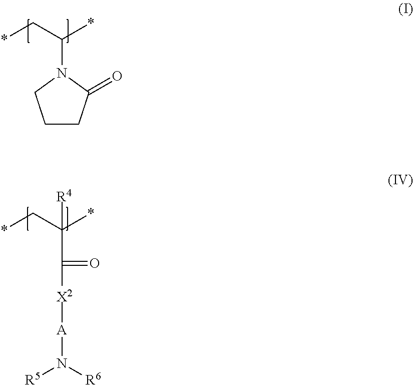

in which
R⁴ is a hydrogen atom or a methyl group,
X² is an oxygen atom or an NH group,
A is a (C₂ to C₃) alkylene group (in particular ethane-1,2-diyl or propane-1,3-diyl),
R⁵ and R⁶ independently of one another are each a (C₁ to C₄) alkyl group
and
(d) a cationic surfactant, selected from N—(C₁₂ to C₂₂) alkyl-N,N,N-trimethylammonium compounds.

(G):

An agent for treating keratin-containing fibers, especially human hair, comprising in a cosmetic carrier

(a) about 0.1 to about 3.0 wt % of a cationic cellulose compound of the INCI name Polyquaternium-4
and
(b) about 0.1 to about 10.0 wt % of a copolymer of the INCI name Vinylcaprolactam/VP/Dimethylaminoethyl-methacrylate copolymer
and
(c) about 0.01 to about 5.0 wt % of a copolymer selected from a copolymer of the group that is formed of copolymers of the INCI name Polyquaternium-11 and copolymers of the INCI name VP/DMAPA Acrylates Copolymer and
(d) a cationic surfactant, selected from N—(C₁₂ to C₂₂) alkyl-N,N,N-trimethylammonium compounds.

(H):

An agent for treating keratin-containing fibers, especially human hair, comprising in a cosmetic carrier

(a) about 0.8 to about 1.2 wt % of a cationic cellulose compound of the INCI name Polyquaternium-4
and
(b) about 0.5 to about 5.0 wt % of a copolymer of the INCI name Vinylcaprolactam/VP/Dimethylaminoethylmethacrylate copolymer
and
(c) about 0.15 to about 1.5 wt % of a copolymer selected from a copolymer of the group that is formed of copolymers of the INCI name Polyquaternium-11 and copolymers of the INCI name VP/DMAPA Acrylates Copolymer and
(d) a cationic surfactant selected from N—(C₁₂ to C₂₂) alkyl-N,N,N-trimethylammonium compounds.

(I):

An agent for treating keratin-containing fibers, especially human hair, comprising in a cosmetic carrier

(a) about 0.1 to about 3.0 wt % of a cationic cellulose compound
and
(b) about 0.1 to about 10 wt % of a copolymer containing a structural unit of the Formula (I) and a structural unit of the Formula (II) and a structural unit of the Formula (III)

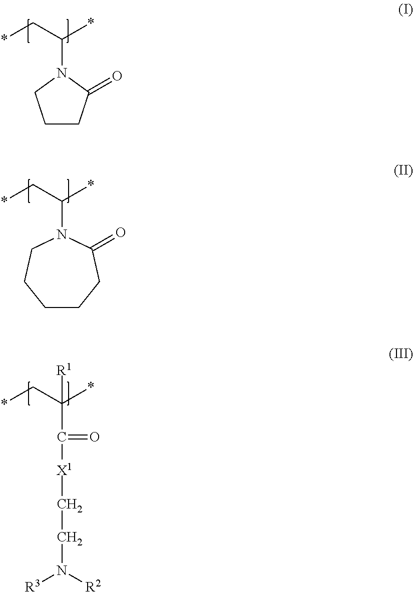

R¹ is a hydrogen atom or a methyl group,
X¹ is an oxygen atom or an NH group,
R² and R³ independently of one another are each a (C₁ to C₄) alkyl group
and
(c) about 0.01 to about 5.0 wt % of a copolymer containing a structural unit of the Formula (I) and a structural unit of the Formula (IV)

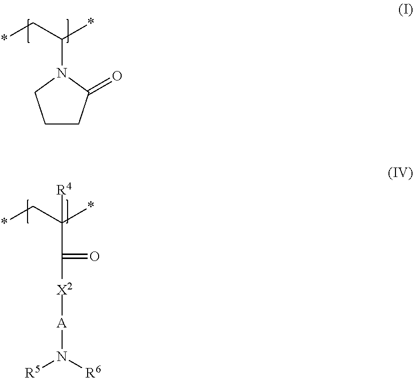

in which
R⁴ is a hydrogen atom or a methyl group,
X² is an oxygen atom or an NH group,
A is a (C₂ to C₃) alkylene group (in particular ethane-1,2-diyl or propane-1,3-diyl),
R⁵ and R⁶ independently of one another are each a (C₁ to C₄) alkyl group,
and
(d) a cationic surfactant, selected from N—(C₁₂ to C₂₂) alkyl-N,N,N-trimethylammonium compounds
and
(e) a cationic surfactant according to Formula (VI),

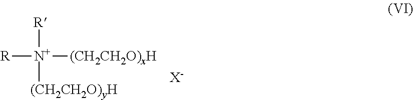

in which
x and y independently of one another are each a whole number greater than 0,
R is a (C₈ to C₂₀) alkyl group or a (C₈ to C₂₀) alkenyl group,
R′ is a *—(CH₂CH₂O)_{z}H group in which z is a whole number greater than 0, a (C₈ to C₂₀) alkyl group or a (C₈ to C₂₀) alkenyl group, and
X⁻ is a physiologically compatible anion.

(J):

An agent for treating keratin-containing fibers, especially human hair, comprising in a cosmetic carrier

(a) about 0.8 to about 1.2 wt % of a cationic cellulose compounds
and
(b) about 0.5 to about 5.0 wt % of a copolymer containing a structural unit of the Formula (I) and a structural unit of the Formula (II) and a structural unit of the Formula (III)

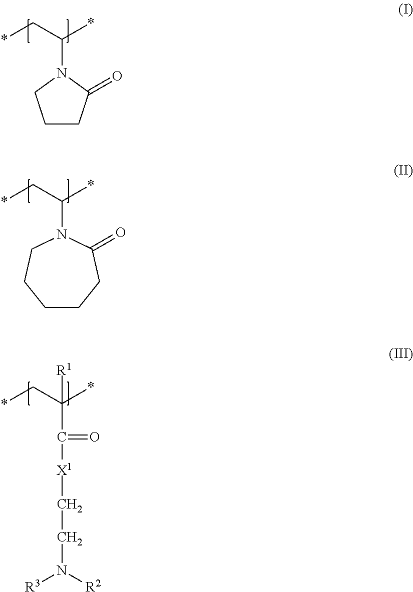

in which
R¹ is a hydrogen atom or a methyl group,
X¹ is an oxygen atom or an NH group,
R² and R³ independently of one another are each a (C₁ to C₄) alkyl group
and
(c) about 0.15 to about 1.5 wt % of a copolymer containing a structural unit of the Formula (I) and a structural unit of the Formula (IV)

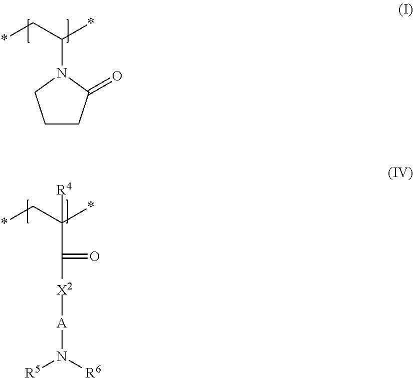

in which
R⁴ is a hydrogen atom or a methyl group,
X² is an oxygen atom or an NH group,
A is a (C₂ to C₃) alkylene group (in particular ethane-1,2-diyl or propane-1,3-diyl),
R⁵ and R⁶ independently of one another are each a (C₁ to C₄) alkyl group
and
(d) a cationic surfactant selected from N—(C₁₂ to C₂₂) alkyl-N,N,N-trimethylammonium compounds
and
(e) a cationic surfactant according to Formula (VI),

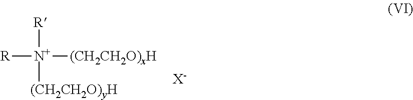

in which
x and y independently of one another are each a whole number greater than 0,
R is a (C₈ to C₂₀) alkyl group or a (C₈ to C₂₀) alkenyl group,
R′ is a *—(CH₂CH₂O)_{z}H group in which z is a whole number greater than 0, a (C₈ to C₂₀) alkyl group or a (C₈ to C₂₀) alkenyl group,
X⁻ is a physiologically compatible anion.

(K):

An agent for treating keratin-containing fibers, especially human hair, comprising in a cosmetic carrier

(a) about 0.1 to about 3.0 wt % of a cationic cellulose compound of the INCI name Polyquaternium-4
and
(b) about 0.1 to about 10.0 wt % of a copolymer of the INCI name Vinylcaprolactam/VP/Dimethylaminoethyl-methacrylate copolymer
and
(c) about 0.01 to about 5.0 wt % of a copolymer selected from a copolymer of the group that is formed of copolymers of the INCI name Polyquaternium-11 and copolymers of the INCI name VP/DMAPA Acrylates Copolymer and
(d) a cationic surfactant selected from N—(C₁₂ to C₂₂) alkyl-N,N,N-trimethylammonium compounds
and
(e) a cationic surfactant according to Formula (VI),

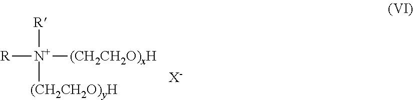

in which
x and y independently of one another are each a whole number greater than 0,
R is a (C₈ to C₂₀) alkyl group or a (C₈ to C₂₀) alkenyl group,

R′ is a *—(CH₂CH₂O)_{z}H group in which z is a whole number greater than 0, a (C₈ to C₂₀) alkyl group or a (C₈ to C₂₀) alkenyl group, and

X⁻ is a physiologically compatible anion.

(L):

An agent for treating keratin-containing fibers, especially human hair, comprising in a cosmetic carrier

(a) about 0.8 to about 1.2 wt % of a cationic cellulose compound of the INCI name Polyquaternium-4 and
(b) about 0.5 to about 5.0 wt % of a copolymer of the INCI name Vinylcaprolactam/VP/Dimethylaminoethylmethacrylate copolymer
and
(c) about 0.15 to about 1.5 wt % of a copolymer selected from a copolymer of the group that is formed of copolymers of the INCI name Polyquaternium-11 and copolymers of the INCI name VP/DMAPA Acrylates Copolymer and
(d) a cationic surfactant selected from N—(C₁₂ to C₂₂) alkyl-N,N,N-trimethylammonium compounds
and

### (e) Quaternium-52.

The inventive agents can optionally additionally comprise an additional setting polymer that differs from the polymers (a), (b) and (c), with the proviso that their addition does not reduce the effect of the inventive polymer combination. In one embodiment, the optionally added setting polymers are cationic and/or non-ionic.

Setting polymers contribute to the hold and/or to the creation of the hair volume and the hair body of the whole hairstyle. These polymers are also film-forming polymers at the same time and therefore in general are typical substances for hair styling treatment agents such as hair sets, hair foams, hair waxes, and hair sprays. The film formation can be in completely selected areas and bond only some fibers together.

The so-called curl-retention test is frequently used as a test method for the setting action.

Film-forming polymers are understood to mean those polymers that on drying leave a continuous film on the skin, the hair or the nails. These types of film-formers can be used in the widest variety of cosmetic products such as, for example make up masks, make up, hair sets, hair sprays, hair gels, hair waxes, hair conditioners, shampoos or nail varnishes. Those polymers are particularly suitable, which are sufficiently soluble in alcohol or water/alcohol mixtures, such that they are present in completely dissolved form in the agent contemplated herein. The film-forming polymers can be of synthetic or of natural origin.

According to an exemplary embodiment, film-forming polymers are further understood to mean those polymers that, when used in concentrations of about 0.01 to about 20 wt % in aqueous, alcoholic or aqueous alcoholic solution, are able to precipitate out a transparent polymer film on the hair.

Suitable setting cationic polymers are commercially available for example as
Copolymers of methacryloylaminopropyllauryldimethylammonium chloride with N-vinyl pyrrolidone and dimethylaminopropylmethacrylamide with the INCI name Polyquaternium-55 under the trade names Styleze® W-10, Styleze® W-20 (ISP),
Copolymers of methacryloylaminopropyllauryldimethylammonium chloride with N-vinyl pyrrolidone, N-vinyl caprolactam and dimethylaminopropylmethacrylamide with the INCI name Polyquaternium-69 under the trade names Aquastyle® 300 (ISP),
Copolymers of N-methylvinyl imidazole and vinyl pyrrolidone with the INCI name Polyquaternium-16 under the trade names Luviquat® Style, Luviquat® FC 370, Luviquat® FC 550, Luviquat® FC 905 and Luviquat® HM 552, or as Polyquaternium-44 under the trade name Luviquat® UltraCare from BASF SE,
Copolymers of N-methylvinyl imidazole and vinyl pyrrolidone and vinyl caprolactam copolymers with the INCI name Polyquarternium-46 and under the trade name Luviquat® Hold from BASF SE, and
Copolymers of N-methylvinyl imidazole and vinyl pyrrolidone and vinyl imidazole and methacrylamide with the INCI name Polyquaternium-68 and under the trade name Luviquat® Supreme from BASF SE.
   As used herein, a non-ionic polymer is understood to mean a polymer that in a protic solvent under standard conditions carries essentially no structural units containing cationic or anionic groups that have to be compensated by counter ions in order to maintain electroneutrality. Cationic groups include for example quaternized ammonium groups but no protonated amines. Anionic groups include for example carboxylic acid and sulfonic acid groups.

Suitable setting non-ionic polymers are for example
polyvinyl pyrrolidone,
copolymers of N-vinyl pyrrolidone and vinyl esters of carboxylic acids containing 2 to 18 carbon atoms, especially from N-vinyl pyrrolidone and vinyl acetate,
copolymers of N-vinyl pyrrolidone and N-vinyl imidazole and methacrylamide,
copolymers of N-vinyl pyrrolidone and N-vinyl imidazole and acrylamide.

It has proved particularly suitable when the agents contemplated herein additionally comprise a non-ionic surfactant.

Non-ionic surfactants comprise e.g. a polyol group, a polyalkylene glycol ether group or a combination of polyol ether groups and polyglycol ether groups as the hydrophilic group. Exemplary compounds of this type are
addition products of 2 to 100 moles ethylene oxide and/or 1 to 5 moles propylene oxide to linear and branched fatty alcohols containing 8 to 30 carbon atoms, to fatty acids containing 8 to 30 carbon atoms and to alkyl phenols containing 8 to 15 carbon atoms in the alkyl group,
methyl or C₂-C₆ alkyl group end blocked addition products of 2 to 50 moles ethylene oxide and/or 1 to 5 moles propylene oxide to linear and branched fatty alcohols with 8 to 30 carbon atoms, to fatty acids with 8 to 30 carbon atoms and to alkyl phenols with 8 to 15 carbon atoms in the alkyl group, such as, for example, the commercially available types Dehydrol® LS, Dehydrol® LT (Cognis),
C₁₂-C₃₀ fatty acid mono and diesters of addition products of 1 to 30 moles ethylene oxide to glycerin,
addition products of 5 to 60 moles ethylene oxide to castor oil and hydrogenated castor oil, polyol esters of fatty acids, such as, for example, the commercial product Hydagen® HSP (Cognis) or Sovermol types (Cognis),
alkoxylated triglycerides,
alkoxylated fatty acid alkyl esters of the Formula (E4-I)

R¹CO—(OCH₂CHR²)_{w}OR³ (E4-I)
in which R¹CO is a linear or branched, saturated and/or unsaturated acyl group containing 6 to 22 carbon atoms, R² is hydrogen or methyl, R³ is linear or branched alkyl groups containing 1 to 4 carbon atoms and w is numbers from 1 to 20,
amine oxides,
mixed hydroxy ethers, such as are described for example in DE-OS 1 973 8866,
sorbitol esters of fatty acids and addition products of ethylene oxide to sorbitol esters of fatty acids such as e.g. the polysorbates,
sugar esters of fatty acids and addition products of ethylene oxide to sugar esters of fatty acids,
addition products of ethylene oxide to fatty acid alkanolamides and fatty amines,
sugar surfactants of the type alkyl and alkenyl oligoglycosides according to Formula (E4-II),

R⁴O-[G]ₚ (E4-II)
in which R⁴ is an alkyl or alkenyl group containing 4 to 22 carbon atoms, G is a sugar group containing 5 or 6 carbon atoms and p is numbers from 1 to 10. They can be obtained according to the appropriate methods of preparative organic chemistry.

Alkylene oxide addition products to saturated, linear fatty alcohols and fatty acids, each with 2 to 100 moles ethylene oxide per mole fatty alcohol or fatty acid, have proved to be quite suitable non-ionic surfactants. Similarly, preparations with excellent properties are obtained when they comprise C₁₂-C₃₀ fatty acid mono and diesters of addition products of 1 to 30 moles ethylene oxide to glycerin and/or addition products of 5 to 60 moles ethylene oxide to castor oil and hydrogenated castor oil as the non-ionic surfactants. The addition products of 5 to 60 mol ethylene oxide on castor oil, hydrogenated castor oil are very suitable.

In an embodiment, the agents exhibit a pH of from about 2 to about 11. In another embodiment, the pH range is from about 2 to about 8. In the context herein, the pH data refer to the pH at 25° C. unless otherwise stated.

The agents according to another embodiment can additionally comprise the auxiliaries and additives that are usually incorporated into conventional styling agents.

In particular, additional care products may be mentioned as suitable auxiliaries and additives.

A silicone oil and/or a silicone gum for example, can be employed as the care substance.

Suitable silicone oils or silicone gums include dialkyl and alkylarylsiloxanes, such as, for example dimethylpolysiloxane and methylphenylpolysiloxane, as well as their alkoxylated, quaternized or also anionic derivatives. Cyclic and linear polydialkylsiloxanes, their alkoxylated and/or aminated derivatives, dihydroxypolydimethylsiloxanes and polyphenylalkylsiloxanes are preferred.

Silicone oils afford the most varied effects. Thus, for example, they simultaneously influence the dry and wet compatibility, the feel of the dry and wet hair as well as the gloss. The term, “silicone oils” is understood by the person skilled in the art to mean organosilicon compounds with a plurality of structures. In the first instance they include the dimethiconols.

The following commercial products are given as examples of such products: Botanisil NU-150M (Botanigenics), Dow Corning 1-1254 Fluid, Dow Corning 2-9023 Fluid, Dow Corning 2-9026 Fluid, Ultrapure Dimethiconol (Ultra Chemical), Unisil SF-R (Universal Preserve), X-21-5619 (Shin-Etsu Chemical Co.), Abil OSW 5 (Degussa Care Specialties), ACC DL-9430 Emulsion (Taylor Chemical Company), AEC Dimethiconol & Sodium Dodecylbenzene sulfonate (A & E Connock (Perfumery & Cosmetics) Ltd.), B C Dimethiconol Emulsion 95 (Basildon Chemical Company, Ltd.), Cosmetic Fluid 1401, Cosmetic Fluid 1403, Cosmetic Fluid 1501, Cosmetic Fluid 1401 DC (all from Chemsil Silicones, Inc.), Dow Corning 1401 Fluid, Dow Corning 1403 Fluid, Dow Corning 1501 Fluid, Dow Corning 1784 HVF Emulsion, Dow Corning 9546 Silicone Elastomer Blend (all from Dow Corning Corporation), Dub Gel SI 1400 (Stearinerie Dubois Fils), HVM 4852 Emulsion (Crompton Corporation), Jeesilc 6056 (Jeen International Corporation), Lubrasil, Lubrasil DS (both from Guardian Laboratories), Nonychosine E, Nonychosine V (both from Exsymol), SanSurf Petrolatum-25, Satin Finish (both from Collaborative Laboratories, Inc.), Silatex-D30 (Cosmetic Ingredient Resources), Silsoft 148, Silsoft E-50, Silsoft E-623 (all from Crompton Corporation), SM555, SM2725, SM2765, SM2785 (all from GE Silicones), Taylor T-Sil CD-1, Taylor TME-4050E (all from Taylor Chemical Company), TH V 148 (Crompton Corporation), Tixogel CYD-1429 (Sud-Chemie Performance Additives), Wacker-Belsil CM 1000, Wacker-Belsil CM 3092, Wacker-Belsil CM 5040, Wacker-Belsil DM 3096, Wacker-Belsil DM 3112 VP, Wacker-Belsil DM 8005 VP, Wacker-Belsil DM 60081 VP (all from Wacker-Chemie GmbH).

Dimethicones form the second group of the silicones that can be used herein. They can be both linear as well as branched as well as cyclic or cyclic and branched.

Dimethicone copolyols form a further group of suitable silicones. Suitable dimethicone copolyols are commercially available and are marketed for example by Dow Corning under the trade name Dow Corning® 5330 Fluid.

Naturally, the teaching herein also includes the fact that the dimethiconols, dimethicones and/or dimethicone copolymers can already be present as an emulsion. The corresponding emulsions of the dimethiconols, dimethicones and/or dimethicone copolyols can be produced both after the production of the corresponding dimethiconols, dimethicones and/or dimethicone copolyols from these and the usual emulsification processes known to the person skilled in the art. Cationic, anionic, non-ionic or zwitterionic surfactants and emulsifiers can be used as auxiliaries and adjuvants for the production of the corresponding emulsions. Naturally, the emulsions of the dimethiconols, dimethicones and/or dimethicone copolyols can also be produced directly by an emulsion polymerization process. These types of processes are also well known to the person skilled in the art.

When the dimethiconols, dimethicones and/or dimethicone copolyols are used as an emulsion, then in an embodiment, the droplet size of the emulsified particles ranges from about 0.01 to about 10 000 μm, for example about 0.01 to about 100 μm, for example about 0.01 to about 20 μm, such as about 0.01 to about 10 μm. The particle size is determined here according to the light scattering method.

If branched dimethiconols, dimethicones and/or dimethicone copolyols are used, then it can be taken as understood that the branching is greater than a fortuitous branching that accidentally results from impurities in the respective monomers. Accordingly, in the context herein, the degree of branching is understood to be greater than about 0.01% for branched dimethiconols, dimethicones and/or dimethicone copolyols. In an embodiment, the degree of branching is greater than 0.1%, for example greater than about 0.5%. The degree of branching is determined from the ratio of the unbranched monomers to the branched monomers, i.e. the amount of tri- and tetrafunctional siloxanes. According to an embodiment, both low-branched as well as highly branched dimethiconols, dimethicones and/or dimethicone copolyols can be quite suitable.

Further suitable silicones are amino-functional silicones, especially the silicones compiled under the INCI name amodimethicone. Consequently, in an embodiment, the agents contemplated herein additionally comprise an amino-functional silicone. These are understood to be silicones that possess at least one, optionally substituted, amino group. These silicones are designated as amodimethicones according to the INCI nomenclature and are available, for example, in the form of an emulsion as the commercial product Dow Corning® 939 or as the commercial product Dow Corning® 949 in a mixture with a cationic and a non-ionic surfactant.

In one embodiment, those amino functional silicones are employed, which have an amine number above about 0.25 meq/g, for example above about 0.3 meq/g, such as above about 0.4 meq/g. The amine number stands for the milli-equivalents of amine per gram of the amino functional silicone. It can be measured by titration and can also be reported with the unit mg KOH/g.

In another embodiment, the agents comprise the silicones in amounts of about 0.01 wt % to about 15 wt %, for example in amounts of about 0.05 to about 2 wt %, based on the total agent.

The agent can comprise, for example, a protein hydrolyzate and/or one of its derivatives as a care substance of another compound class.

Protein hydrolyzates are product mixtures obtained by acid-, base- or enzyme-catalyzed degradation of proteins (albumins). The term “protein hydrolyzates” is also understood to mean total hydrolyzates as well as individual amino acids and their derivatives as well as mixtures of different amino acids. In an embodiment, the molecular weight of the protein hydrolyzates utilizable herein ranges between about 75, the molecular weight of glycine, and about 200 000, for example the molecular weight is about 75 to about 50 000, such as about 75 to about 20 000 Dalton.

The added protein hydrolyzates can be of both vegetable as well as animal or marine or synthetic origin.

Animal protein hydrolysates are, for example, protein hydrolysates of elastin, collagen, keratin, silk and milk albumin, which can also be present in the form of their salts. Such products are marketed, for example, under the trade names Dehylan® (Cognis), Promois® (Interorgana), Collapuron® (Cognis), Nutrilan® (Cognis), Gelita-Sol® (Deutsche Gelatine Fabriken Stoess & Co), Lexein® (Inolex), Sericin (Pentapharm) and Kerasol® (Croda).

In an embodiment, the agents comprise the protein hydrolyzates in concentrations of about 0.01 wt % to about 20 wt %, for example about 0.05 wt % up to about 15 wt %, such as in amounts of about 0.05 wt % up to about 5 wt %, each based on the total end-use preparation.

The agent contemplated herein can further comprise a vitamin, a provitamin, a vitamin precursor and/or a derivatives thereof as the care substance.

Suitable vitamins, provitamins and vitamin precursors are normally classified in the groups A, B, C, E, F and H.

Retinol (vitamin A1) as well as 3,4-didehydroretinol, (vitamin A2) belong in the group of substances designated as vitamin A. β-carotene is the provitamin of retinol. Examples of suitable vitamin A components are vitamin A acid and its esters, vitamin A aldehyde and vitamin A alcohol as well as its esters such as the palmitate and acetate. In an embodiment, the agents comprise the vitamin A components in amounts of about 0.05 to about 1 wt %, based on the total end use preparation.

The vitamin B group or the vitamin B complex includes inter alia vitamin B1 (thiamine), vitamin B2 (riboflavin), vitamin B3 (nicotinic acid and/or nicotinic acid amide (niacinamide)), vitamin B5 (pantothenic acid, panthenol and pantolactone), vitamin B6 (pyridoxine as well as pyridoxamine and pyridoxal), vitamin C (ascorbic acid), vitamin E (tocopherols, especially α-tocopherol), vitamin F (linoleic acid and/or linolenic acid), vitamin H.

In another embodiment, the agents comprise vitamins, provitamins and vitamin precursors from groups A, B, C, E and H. Panthenol, pantolactone, pyridoxine and its derivatives as well as nicotinamide and biotin are especially preferred.

D-panthenol is quite suitable as a care substance, optionally in combination with at least one of the abovementioned silicone derivatives.

Like the addition of glycerin and/or propylene glycol, the addition of panthenol increases the flexibility of the polymer film that is formed when the agent is used. Thus, if a particularly flexible hold is desired, then the agents can comprise panthenol instead of or in addition to glycerin and/or propylene glycol. In an embodiment, the agents comprise panthenol in a quantity of about 0.05 to about 10 wt %, for example about 0.1 to about 5 wt %, each based on the total agent.

The agents contemplated herein can further comprise a plant extract as a care substance.

Usually, these extracts are manufactured by extraction of the whole plant. In individual cases, however, it can also be suitable to produce the extracts solely from blossoms and/or leaves of the plant.

Mainly extracts from green tea, oak bark, stinging nettle, hamamelis, hops, henna, camomile, burdock root, field horsetail, hawthorn, linden flowers, almonds, aloe vera, spruce needles, horse chestnut, sandal wood, juniper, coconut, mango, apricot, lime, wheat, kiwi, melon, orange, grapefruit, sage, rosemary, birch, malva, lady's smock, common yarrow, thyme, lemon balm, rest-harrow, coltsfoot, marshmallow, meristem, ginseng and ginger are suitable.

In addition, mixtures of a plurality, particularly two different plant extracts can be employed in the agents.

Mono or oligosaccharides can also be incorporated as the care substance into the agents contemplated herein.

Both monosaccharides as well as oligosaccharides, such as for example raw sugar, lactose and raffinose, can be incorporated. The use of monosaccharides is preferred. In an embodiment, the monosaccharides include those compounds that contain 5 or 6 carbon atoms.

Suitable pentoses and hexoses are for example ribose, arabinose, xylose, lyxose, allose, altrose, glucose, mannose, gulose, idose, galactose, talose, fucose and fructose. Arabinose, glucose, galactose and fructose are the preferred incorporated carbohydrates; glucose is quite suitably incorporated, and is suitable both in the D(+) or L(−) configuration or as the racemate.

In addition, derivatives of these pentoses and hexoses can also be incorporated, such as the corresponding onic and uronic acids (sugar acids), sugar alcohols, and glycosides. Suitable sugar acids are the gluconic acid, the glucuronic acid, the sugar acids, the mannosugar acids and the mucic acids. Suitable sugar alcohols are sorbitol, mannitol and dulcitol. Suitable glycosides are the methyl glucosides.

As the incorporated mono- and oligosaccharides are usually obtained from natural raw materials such as starch, they generally possess the configurations that correspond to these raw materials (e.g. D-glucose, D-fructose and D-galactose).

In an embodiment, the inventive agents comprise the mono or oligosaccharides in an amount of about 0.1 to about 8 wt %, for example about 1 to about 5 wt %, based on the total end-use preparation.

The agent can further comprise a lipid as a care substance.

Suitable lipids for use herein are phospholipids, for example soy lecithin, egg lecithin and cephalins as well as the substances known under the INCI names Linoleamidopropyl PG-Dimonium Chloride Phosphate, Cocamidopropyl PG-Dimonium Chloride Phosphate and Stearamidopropyl PG-Dimonium Chloride Phosphate. These are commercialized, for example, by the Mona Company under the trade names Phospholipid EFA®, Phospholipid PTC® and Phospholipid SV®. The agents in an embodiment comprise the lipids in amounts of about 0.01 to about 10 wt %, for example about 0.1 to about 5 wt %, based on the total end-use preparation.

Oil bodies are also suitable as a care substance.

The natural and synthetic cosmetic oil bodies include, for example:
vegetal oils. Examples of such oils are sunflower oil, olive oil, soya oil, rapeseed oil, almond oil, jojoba oil, orange oil, wheat germ oil, peach stone oil and the liquid parts of coconut oil. Other triglyceride oils such as the liquid fractions of beef tallow as well as synthetic triglyceride oils are also suitable, however.
liquid paraffin oils, isoparaffin oils and synthetic hydrocarbons as well as di-n-alkyl ethers containing a total of 12 to 36 carbon atoms, particularly 12 to 24 carbon atoms such as, for example, di-n-octyl ether, di-n-decyl ether, di-n-nonyl ether, di-n-undecyl ether, di-n-dodecyl ether, n-hexyl n-octyl ether, n-octyl n-decyl ether, n-decyl n-undecyl ether, n-undecyl n-dodecyl ether and n-hexyl n-undecyl ether and di-tert.butyl ether, diisopentyl ether, di-3-ethyldecyl ether, tert.butyl n-octyl ether, isopentyl n-octyl ether and 2-methylpentyl n-octyl ether. The commercial products 1,3-di-(2-ethylhexyl)cyclohexane (Cetiol® S) and di-n-octyl ether (Cetiol® OE) are suitable.
ester oils. Ester oils are understood to mean the esters of C₆-C₃₀ fatty acids with C₂-C₃₀ fatty alcohols. Monoesters of fatty acids with alcohols having 2 to 24 carbon atoms are preferred. Isopropyl myristate (Rilanit® IPM), isononanoic acid C₁₆₋₁₈ alkyl ester (Cetiol® SN), 2-ethylhexyl palmitate (Cegesoft® 24), stearic acid 2-ethylhexyl ester (Cetiol® 868), cetyl oleate, glycerin tricaprylate, cocofatty alcohol caprinate/-caprylate (Cetiol® LC), n-butyl stearate, oleyl erucate (Cetiol® J 600), isopropyl palmitate (Rilanit® IPP), oleyl oleate (Cetiol®), lauric acid hexyl ester (Cetiol® A), di-n-butyl adipate (Cetiol® B), myristyl myristate (Cetiol® MM), cetearyl isononanoate (Cetiol® SN), and oleic acid decyl ester (Cetiol® V) are particularly suitable.
dicarboxylic acid esters such as di-n-butyl adipate, di-(2-ethylhexyl) adipate, di-(2-ethylhexyl) succinate and di-isotridecyl acetate as well as diol esters such as ethylene glycol dioleate, ethylene glycol di-isotridecanoate, propylene glycol di(2-ethylhexanoate), propylene glycol di-isostearate, propylene glycol di-pelargonate, butane diol di-isostearate, neopentyl glycol dicaprylate,
symmetrical, unsymmetrical or cyclic esters of carbon dioxide with fatty alcohols, e.g. as described in DE-OS 197 56 454, glycerine carbonate or dicaprylyl carbonate (Cetiol® CC),
trifatty acid esters of saturated and/or unsaturated linear and/or branched fatty acids with glycerin,
fatty acid partial glycerides, under which are understood monoglycerides, diglycerides and their industrial mixtures. When using industrial products, minor amounts of triglycerides may still be contained as a result of the production process. The partial glycerides preferably comply with the Formula (D4-I),

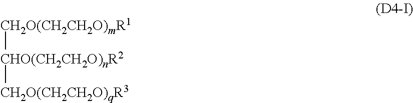

in which R¹, R² and R³ independently of each other are hydrogen or a linear or branched, saturated and/or unsaturated acyl group containing 6 to 22 carbon atoms, such as 12 to 18 carbon atoms, with the proviso that at least one of these groups is an acyl group and at least one of these groups is hydrogen. The sum of (m+n+q) is 0 or numbers from 1 to 100, such as 0 or 5 to 25. Preferably, R¹ is an acyl group and R² and R³ is hydrogen and the sum of (m+n+q) is 0. Typical examples are mono- and/or diglycerides based on caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, arachidonic acid, gadoleic acid, behenic acid and erucic acid as well as their industrial mixtures. Oleic acid monoglycerides are suitably employed.

In an embodiment, the added quantity of the natural and synthetic cosmetic oil bodies in the agents is about 0.1 to about 30 wt %, based on the total end-use preparation, for example about 0.1 to about 20 wt %, such as about 0.1 to about 15 wt %.

Although each of the cited care substances alone already provides a satisfactory result, in the context herein all embodiments are also included, in which the agent comprises a plurality of care substances even from different groups.

By the addition of a UV filter, both the agent itself as well as the treated fibers can be protected against damage from UV radiation. Consequently, in another embodiment, a UV filter is added to the agent. The suitable UV filters are not generally limited in regard to their structure and their physical properties. Indeed, all UV filters that can be employed in the cosmetic field having an absorption maximum in the UVA (315-400 nm), in the UVB (280-315 nm) or in the UVC (<280 nm) regions are suitable. UV filters having an absorption maximum in the UVB region, especially in the range from about 280 to about 300 nm, are particularly suitable.

Particularly suitable UV-filters are chosen from substituted benzophenones, p-aminobenzoates, diphenylacrylates, cinnamates, salicylates, benzimidazoles and o-aminobenzoates.

In an embodiment, the agent comprises the UV filters in quantities of about 0.01 to about 5 wt %, based on the total end-use preparation. Quantities of about 0.1 to about 2.5 wt % are preferred.

In a particular embodiment, the agent contemplated herein further comprises a substantive dye. Application of the agent then enables the treated keratinic fiber not only to be temporarily styled but also to be dyed at the same time. This can be particularly desirable when only a temporary dyeing is desired, for example with flamboyant fashion colors that can be subsequently removed from the keratinic fibers by simply washing them out.

Substantive dyes are usually nitrophenylenediamines, nitroamino phenols, azo dyes, anthraquinones or indophenols. Preferred substantive dyestuffs are the compounds known under the international designations or trade names HC Yellow 2, HC Yellow 4, HC Yellow 5, HC Yellow 6, HC Yellow 12, Acid Yellow 1, Acid Yellow 10, Acid Yellow 23, Acid Yellow 36, HC Orange 1, Disperse Orange 3, Acid Orange 7, HC Red 1, HC Red 3, HC Red 10, HC Red 11, HC Red 13, Acid Red 33, Acid Red 52, HC Red BN, Pigment Red 57:1, HC Blue 2, HC Blue 11, HC Blue 12, Disperse Blue 3, Acid Blue 7, Acid Green 50, HC Violet 1, Disperse Violet 1, Disperse Violet 4, Acid Violet 43, Disperse Black 9, Acid Black 1, and Acid Black 52 known compounds as well as 1,4-diamino-2-nitrobenzene, 2-amino-4-nitrophenol, 1,4-bis(β-hydroxyethyl)amino-2-nitrobenzene, 3-nitro-4(β-hydroxyethyl)aminophenol, 2-(2′-hydroxyethyl)amino-4-6-dinitrophenol, 1-(2′-hydroxyethyl)amino-4-methyl-2-nitrobenzene, 1-amino-4-(2′-hydroxyethyl)-amino-5-chloro-2-nitrobenzene, 4-amino-3-nitrophenol, 1-(2′-ureidoethyl)amino-4-nitrobenzene, 4-amino-2-nitrodiphenylamine-2′-carboxylic acid, 6-nitro-1,2,3,4-tetrahydroquinoxaline, 2-hydroxy-1,4-naphthoquinone, picramic acid and its salts, 2-amino-6-chloro-4-nitrophenol, 4-ethylamino-3-nitrobenzoic acid and 2-chloro-6-ethylamino-1-hydroxy-4-nitrobenzene. Cationic substantive dyes are suitably employed. Particular preference is given here to

(a) cationic triphenylmethane dyes, such as, for example, Basic Blue 7, Basic Blue 26, Basic Violet 2 and Basic Violet 14,
(b) aromatic systems which are substituted by a quaternary nitrogen group, such as, for example, Basic Yellow 57, Basic Red 76, Basic Blue 99, Basic Brown 16 and Basic Brown 17, and
(c) substantive dyes, which comprise a heterocycle that has at least one quaternary nitrogen atom, as are specified, for example, in EP-A2-998 908 in the claims 6 to 11 which is explicitly incorporated here by reference.

The dyes, which are also known under the names Basic Yellow 87, Basic Orange 31 and Basic Red 51, are quite suitable cationic substantive dyes of group (c). The cationic substantive dyes that are commercialized under the trade name Arianor® are likewise quite particularly suitable cationic substantive dyes as used herein.

The inventive agents according to this embodiment comprise the substantive dyes in a quantity of about 0.001 to about 20 wt %, based on the total agent.

In a further embodiment, the agents contemplated herein are exempt from oxidation dye precursors. Oxidation dye precursors are divided into so-called developer components and coupler components. Under the influence of oxidizing agents or from atmospheric oxygen, the developer components form the actual colorants among each other or by coupling with one or more coupler components.

The formulation of the inventive agents can be in all usual forms for styling agents, for example, in the form of solutions that can be applied as hair water or pump or aerosol spray onto the hair, in the form of creams, emulsions, waxes, gels or also surfactant-containing foaming solutions or other preparations, which are suitable for application on the hair.

Hair creams and hair gels generally comprise structurants and/or thickening polymers, which lend the desired consistency to the products. Structurants and/or thickening polymers are typically added in amounts of about 0.1 to about 10 wt %, based on the total product. One embodiment provides for the use of quantities of about 0.5 to about 5 wt %, particularly about 0.5 to about 3 wt %.

In another embodiment, the inventive agent is a pump spray, an aerosol spray, a pump foam or an aerosol foam.

For this the agents are packed in a dispensing device, illustrated by either a pressurized gas container additionally containing a propellant (“aerosol container”) or by a non-aerosol container.

The pressurized gas containers, by which a product is dispersed through a valve by means of the internal gas pressure in the container, are defined as “aerosol containers”. The opposite of the aerosol definition, a container under normal pressure, is defined as a “non-aerosol container”, from which a product is dispersed by the mechanical actuation of a pump system.

The agents contemplated herein, in an embodiment, are packed as an aerosol hair foam or aerosol hair spray. Consequently, the agents additionally comprise at least one propellant.

Inventively suitable exemplary propellants are selected from N₂O, dimethyl ether, CO₂, air, alkanes containing 3 to 5 carbon atoms, such as propane, n-butane, iso-butane, n-pentane and iso-pentane, and their mixtures. Dimethyl ether, propane, n-butane, iso-butane and their mixtures are particularly suitable.

According to an embodiment, the cited alkanes, mixtures of the cited alkanes or mixtures of the cited alkanes with dimethyl ether are added as the sole propellant. However, the joint utilization with propellants of the fluorochlorohydrocarbon type, but especially fluorinated hydrocarbons, may also be used.

In regard to the weight ratio of propellant to the usual ingredients of the preparation, the size of the aerosol droplets or the foam bubbles and the relevant size distribution can be adjusted for a given spray device.

The quantity of added propellant varies as a function of the actual composition of the agent, the packaging used and the desired product type, for example hair spray or hair foam. In an embodiment, when a conventional spray device is used, aerosol foam products comprise the propellant in amounts of about 1 to about 35 wt %, based on the total product. Quantities of about 2 to about 30 wt %, for example about 3 to about 15 wt %, are particularly suitable. Aerosol sprays generally comprise greater amounts of propellant. In this embodiment, the propellant is added in amounts of about 30 to about 98 wt %, based on the total product. Quantities of about 40 to about 95 wt %, for example about 50 to about 95 wt %, are particularly suitable.

The aerosol products can be manufactured according to conventional techniques. Generally, all ingredients of the agent, excepting the propellant, are charged into a suitable pressure-resistant container. This is thereupon sealed with a valve. The desired quantity of propellant is then filled by means of conventional techniques.

Agents that are in the form of gels are foamed in a two-chamber aerosol container, preferably with isopentane as the propellant, which is incorporated into the agent contemplated herein and packed in the first chamber of the two-chamber aerosol container. At least one additional propellant that differs from isopentane is packed in the second chamber of the two-chamber aerosol container and generates a higher pressure than the isopentane. In an embodiment, the propellants of the second chamber are selected from N₂O, dimethyl ether, CO₂, air, alkanes containing 3 or 4 carbon atoms (such as propane, n-butane, iso-butane) as well as mixtures thereof.

In another embodiment, the use of the agent contemplated herein is provided for the temporary shaping of hair and/or for lending volume.

The agents contemplated herein and products that comprise these agents, especially aerosol hair foams or aerosol hair sprays, are particularly characterized in that they lend to the treated hair a very strong, long-lasting volume, although the hair remains flexible. If the agent is made up as a hair foam, then a stable, micro-porous and creamy foam is formed that can be uniformly dispersed on the hair without dripping. No visible residues are formed on the hair.

In a further embodiment, a method for treating keratin-containing fibers, especially human hair, includes foaming an agent as contemplated above to a foam by the use of a dispensing device, and the resulting foam is applied onto the keratin-containing fibers.

In an embodiment, the keratin-containing fibers are shaped and this shape is fixed by the agent contemplated herein.

The abovementioned dispensing devices (see above) are suitable for use.

In yet another embodiment, a method for treating keratin-containing fibers, especially human hair, includes applying an agent contemplated herein as a spray onto the keratin-containing fibers by the use of a dispensing device.

In an embodiment, the keratin-containing fibers are shaped and this shape is fixed by the agent contemplated herein.

The abovementioned dispensing devices (see above) are suitable for use.

The following examples are intended to illustrate the subject matter herein in more detail, without limiting it in any way.

### EXAMPLES

All quantities are given as weight percent unless otherwise stated. The following raw materials were used to prepare the formulation shown below:
1 trimethylhexadecylammonium chloride (ca. 24-26% active substance; INCI name: Aqua (Water), Cetrimonium Chloride) (Cognis)
2 tris-(oligooxyethyl)alkylammonium phosphate (ca. 49-51% active substance in water; INCI name: Aqua (Water), Quaternium-52, Propylene Glycol) (Cognis)
3 quaternized cellulose derivative (INCI name: Polyquaternium-4) (National Starch)
4 dimethylaminoethyl methacrylate-vinyl pyrrolidone copolymer, quaternized with diethyl sulfate (ca. 19% solids in water; INCI name: Polyquaternium-11); (ISP)
5 INCI name: Vinylcaprolactam/VP/Dimethyl-aminoethylmethacrylate copolymer (in powder form; ISP)

TABLE 1
Formulation

| Raw Material | E1 |
|---|---|
| Lactic acid | 0.04 |
| Sodium benzoate | 0.3 |
| D-Panthenol | 0.2 |
| Dehyquart A CA ¹ | 1.0 |
| Dehyquart SP ² | 0.5 |
| PEG-40 Hydrogenated castor oil | 0.2 |
| Celquat L-200 ³ | 1.0 |
| Gafquat 755N-PW ⁴ | 6.0 |
| Advantage S ⁵ | 2.5 |
| Water, fully deionized | ad 100 |
| Propane/butane | 8.0 |

The ingredients of the formulation were each filled into an aerosol container that meets the following technical parameter: aluminum reservoir container with valve product 522983 PV 10697 from the Precision Company (Deutsche Prazisions-Ventil GmbH).
The formulation was brought out of the aerosol container as a hair foam and dispersed onto the hair of subjects. The formulation lent a high hold and volume to the hairstyle of the subjects (also controllable by carrying out a high humidity curl retention test). Furthermore, even hours after the application there was no formation of film plaques. Even after brushing, no film plaques were observed.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment, it being understood that various changes may be made in the function and arrangement of elements described in an exemplary embodiment without departing from the scope of the invention as set forth in the appended claims and their legal equivalents.

1. An agent for treating keratin-containing fibers, especially human hair, comprising in a cosmetic carrier:
(a) a cationic cellulose compound;
(b) a copolymer containing a structural unit of Formula (I), a structural unit of Formula (II), and a structural unit of Formula (III) 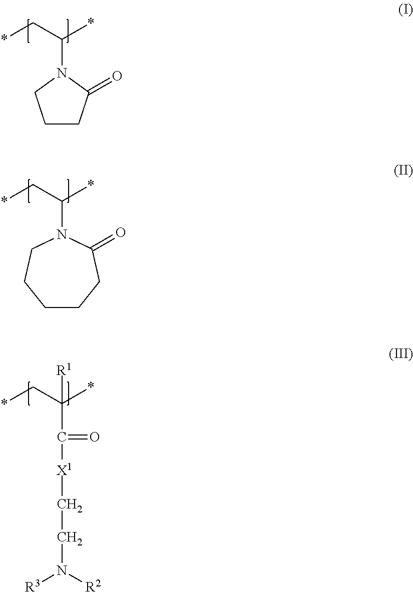
in which
R¹ is a hydrogen atom or a methyl group,
X¹ is an oxygen atom or an NH group,
R² and R³ independently of one another are each a (C₁ to C₄) alkyl group;
and
(c) a copolymer containing a structural unit of Formula (I) and a structural unit of Formula (IV) 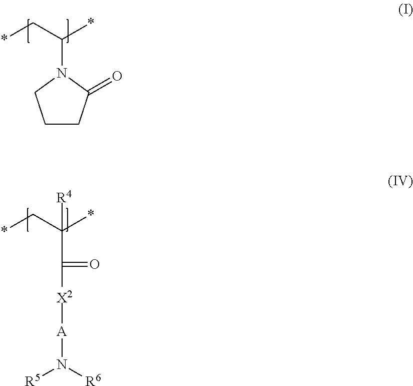
in which
R⁴ is a hydrogen atom or a methyl group,
X² is an oxygen atom or an NH group,
A is a (C₂ to C₃) alkylene group, and
R⁵ and R⁶ independently of one another are each a (C₁ to C₄) alkyl group.

2. The agent according to claim 1, wherein A is ethane-1,2-diyl or propane-1,3-diyl.

3. The agent according to claim 1, wherein the cationic cellulose compound in at least one side chain carries more than one permanent cationic charge.

4. The agent according to claim 1, wherein the cationic cellulose compound is present in an amount of about 0.1 wt % to about 3.0 wt % relative to the weight of the agent.

5. The agent according to claim 4, wherein the cationic cellulose compound is present in an amount of about 0.4 wt % to 1.8 wt % relative to the weight of the agent.

6. The agent according to claim 5, wherein the cationic cellulose compound is present in an amount of about 0.8 wt % to 1.2 wt %, relative to the weight of the agent.

7. The agent according to claim 1, wherein the copolymer (b) comprises a terpolymer obtained from N-vinyl pyrrolidone, N-vinyl caprolactam and N,N-dimethylaminoethyl methacrylate.

8. The agent according to claim 1, wherein the copolymer (b) is present in an amount of about 0.1 wt % to about 10.0 wt %, relative to the weight of the agent.

9. The agent according to claim 8, wherein the copolymer (b) is present in an amount of about 0.5 wt % to 8.0 wt %, relative to the weight of the agent.

10. The agent according to claim 9, wherein the copolymer (b) is present in an amount of about 0.5 wt % to 5.0 wt %, relative to the weight of the agent.

11. The agent according to claim 1, wherein in Formula (IV):
R⁴ is a methyl group;
X² is an oxygen atom or an NH group;
A is ethane-1,2-diyl or propane-1,3-diyl; and
R⁵ and R⁶ independently of one another are each a (C₁ to C₄) alkyl group, with the proviso that:
when X² is an oxygen atom, then A is ethane-1,2-diyl, and
when X² is an NH group, then A is propane-1,3-diyl.

12. The agent according to claim 11, wherein R⁵ and R⁶ are each a methyl group.

13. The agent according to claim 11, wherein X² in Formula (IV) is an oxygen atom and the copolymer (c) additionally comprises a structural unit of Formula (V) 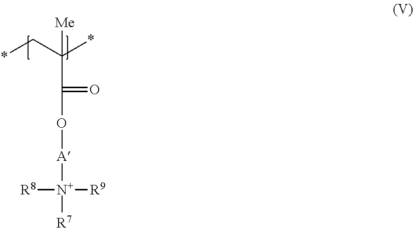
in which
R⁸ and R⁹ independently of one another are each a (C₁ to C₆) alkyl group, A′ is ethane-1,2-diyl or propane-1,3-diyl, and R⁷ is a (C₁ to C₄) alkyl group.

14. The agent according to claim 1, wherein the copolymer (c) is present in an amount of about 0.01 wt % to about 5.0 wt %, relative to the weight of the agent.

15. The agent according to claim 1, wherein the copolymer (c) is present in an amount of about 0.1 wt % to about 2.0 wt %, relative to the weight of the agent.

16. The agent according to claim 1, further comprising a cationic surfactant.

17. The agent according to claim 1, further comprising a propellant.

18. A method for treating keratin-containing fibers, the method comprising the steps of:
foaming an agent to a foam using a dispensing device, the agent comprising in a cosmetic carrier:
(a) a cationic cellulose compound;
(b) a copolymer containing a structural unit of Formula (I), a structural unit of Formula (II), and a structural unit of Formula (III) 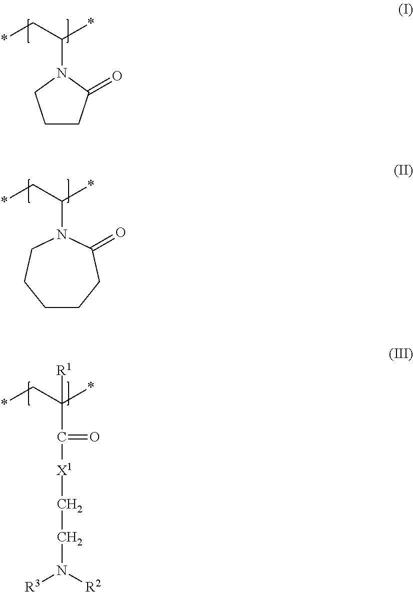
in which
R¹ is a hydrogen atom or a methyl group,
X¹ is an oxygen atom or an NH group,
R² and R³ independently of one another are each a (C₁ to C₄) alkyl group;
and
(c) a copolymer containing a structural unit of Formula (I) and a structural unit of Formula (IV) 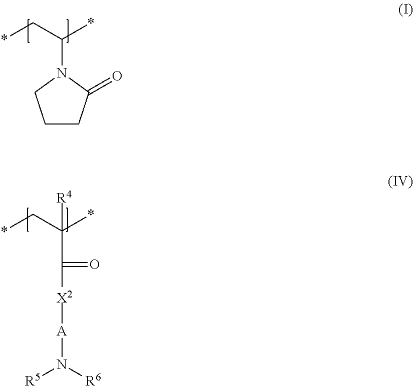
in which
R⁴ is a hydrogen atom or a methyl group,
X² is an oxygen atom or an NH group,
A is a (C₂ to C₃) alkylene group, and
R⁵ and R⁶ independently of one another are each a (C₁ to C₄) alkyl group: and
applying the foam onto the keratin-containing fibers.

19. The method according to claim 18, wherein applying comprises applying the foam onto human hair.

20. A method for treating keratin-containing fibers, the method comprising the step of:
spraying an agent onto the keratin-containing fibers using a dispensing device, the agent comprising in a cosmetic carrier:
(a) a cationic cellulose compound;
(b) a copolymer containing a structural unit of Formula (I), a structural unit of Formula (II), and a structural unit of Formula (III) 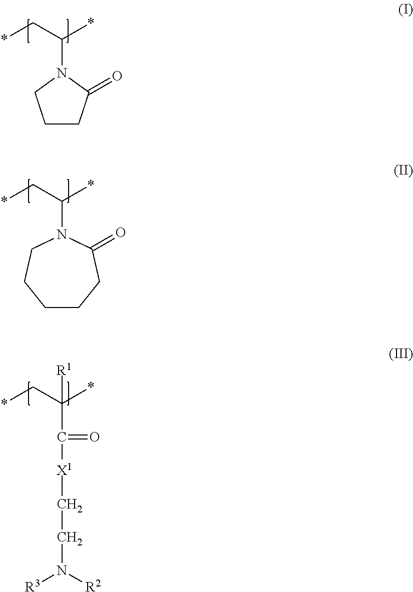
in which
R¹ is a hydrogen atom or a methyl group,
X¹ is an oxygen atom or an NH group,
R² and R³ independently of one another are each a (C₁ to C₄) alkyl group;
and
(c) a copolymer containing a structural unit of Formula (I) and a structural unit of Formula (IV) 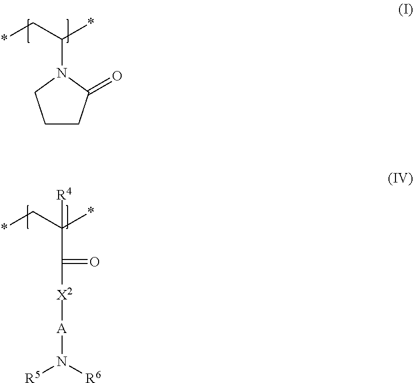
in which
R⁴ is a hydrogen atom or a methyl group,
X² is an oxygen atom or an NH group,
A is a (C₂ to C₃) alkylene group, and
R⁵ and R⁶ independently of one another are each a (C₁ to C₄) alkyl group.
